(19) United States
(12) Patent Application Publication
(10) Pub. No.: US 2024/0110864 A1
(43) Pub. Date: Apr. 4, 2024

(54) PREDICTIVE DIAGNOSTIC TEST FOR EARLY DETECTION AND MONITORING OF DISEASES

(71) Applicant: ONCODEA CORPORATION, Saint Paul, MN (US)

(72) Inventors: Ali Ya KHAMMANIVONG, Saint Paul, MN (US); Dan Que PHAM, Saint Paul, MN (US)

(21) Appl. No.: 18/043,719

(22) PCT Filed: Sep. 1, 2021
(86) PCT No.: PCT/US2021/071334

§ 371 Date: Mar. 1, 2023

Related U.S. Application Data
(60) Provisional application No. 63/073,169, filed on Sep. 1, 2020.

(51) Int. Cl. G01N 21/3577; G01N 21/31; G01N 21/359

(52) U.S. Cl. CPC G01N 21/3577; G01N 21/3103; G01N 21/359; G01N 21/07

(57) ABSTRACT

Methods and system for detection and diagnosis of diseases including cancer. A patient sample is analyzed by absorbance spectroscopy in the near- and mid-infrared range to produce a spectrometric signature, the sample obtained from a patient. The signature is processed by a computational engine using one or more machine learning techniques to determine whether the spectrometric signature indicates the presence of a disease, including one or more cancers. Embodiments operate outside of conventionally accepted wavelength ranges, facilitating more rapid, simple, and reliable testing.

### TECHNICAL FIELD

The present application relates generally to the field of diagnostic tests, and more specifically to diagnosis by means of spectrographic analysis.

### BACKGROUND

Cancer remains a significant health problem worldwide and is the second leading cause of death in the United States. Early cancer detection is still the best weapon against this disease to allow for better treatment options and patient outcomes. Yet, current technologies for cancer screening and early detection are risky, costly, and time-consuming to help win the war against cancer. Cancer screening is recommended only to high-risk populations based on a specific age range, smoking history, alcohol consumption, and potential environmental exposures. Cancer incidence even for those who do not meet these criteria is also rising, such as those with human papillomavirus-associated head and neck cancer. Current tests and procedures used in cancer screening and diagnosis include radiological imaging, endoscopy, biopsy, and cytology.

Tests such as computed tomography (CT), X-ray, and positron emission tomography (PET) carry risks for radiation exposure that can cause cancer in healthy individuals. Endoscopy and biopsy are invasive and can cause discomfort. Since these methods of detecting cancer at an early stage rely on individual behaviors (such as scheduling the mammogram, colonoscopy, or pap smear, etc.), discomforts and risks can create fears and reluctance for getting screened promptly. In addition to being invasive, histopathological diagnoses carry a high probability of false negatives and false positives, resulting in misdiagnosis. This is partially due to certain types of tumors being histologically similar, and due to cells being poorly differentiated which makes it difficult to identify the origin of the tissue.

Various methods of optical spectroscopy have begun to be used in the early diagnosis of cancer, but few have showed promising results. Fourier transform spectroscopy (or FTIR) detection systems have been investigated, and have been used with some promise, using high wavelengths across significant wavelength ranges in the infrared (“IR”) or near-IR band (such as 900-3080 nm) even though many cancer biomarkers often have low sensitivity and low specificity in this wavelength region. However, FTIR spectroscopy has shown promise due to the fact that it provides a simple, rapid, relatively accurate, inexpensive, non-destructive and suitable for automation compared to existing screening, diagnosis, management and monitoring methods. Infrared spectroscopy is an important tool for cancer and other disease screening and detection, because it can help inform decision-making, and improve patient outcomes by providing earlier diagnosis. An example of FTIR spectroscopy is discussed in Kenneth A. Kristoffersen et al., Fourier-transform infrared spectroscopy for monitoring proteolytic reactions using dry-films treated with trifluoroacetic acid, 10 SCI REP 7844 (2020) (available at https://doi.org/10.1038/s41598-020-64583-3).

While near-infrared (NIR) spectroscopy is not a new approach to take in cancer screening, recently published studies encourage the use of higher wavelengths, and a combined approach for measuring multiple types of samples. The range used most frequently for NIR spectroscopy in cancer detection has been 600-1100 nm. Longer wavelengths have a tendency to be mostly absorbed by water, which makes it much more difficult to see any differences. Water, while optically transparent, is highly absorbent below 200 nm and also absorbs some NIR and even more mid-IR and far-IR light.

Additional attempts have been made to utilize liquid biopsy as a means of non-invasive cancer detection. An example of liquid biopsy is shown in U.S. Pat. No. 10,288,615, which describes detecting cancer using blood. Further, studies and disclosures have presented measuring the water content in normal cells versus cancerous cells. An example of an NIR spectroscopy approach is shown, for example, in U.S. Pat. No. 7,706,862, which describes NIR spectral optical imaging using water absorption wavelengths. Spectral optical imaging in the near infrared (NIR) at one or more key water absorption wavelengths is used to identify differences in water content between a region of cancerous or precancerous tissue and a region of normal tissue. Tissue regions in the later stages of cancer have more water content than normal tissues.

The key water absorption “fingerprint” wavelengths include at least one of 980 nm, 1195 nm, 1456 nm, 1944 nm, 2880 nm to 3360 nm, and 4720 nm. In the range of 400 nm to 6000 nm, at least one reference wavelength of low or no water absorption—illustratively, 4500 nm, 2230 nm, 1700 nm, 1300 nm, 1000 nm, and 800 nm is used to produce a reference image to draw a comparison with an image taken at a key water absorption wavelength. Although NIR can be used to measure water absorption by cells, it is not the most reliable technique, nor does it produce clear results each time it is. Because water absorbs much of the wavelength, imaging can be difficult, and accuracy is limited. Molecular fingerprinting involves looking for differences in specific biomarkers that make-up disease fingerprints, and predefined disease biomarkers. A holistic approach to identifying the molecular fingerprints of cancer, for example, is needed to determine not just the existence of a cancer, but the type of cancer. The fingerprints are based on spectrometric signatures using a broad-spectrum spectroscopy, which is a concept well supported by previous studies. Using infrared (IR) spectroscopy, differences in serum components can be documented to generate unique spectrometric signatures specific to different health conditions. However, any previous studies using similar technology have focused on longer wavelength IR absorbance to analyze serum biomolecules. Thus, there exists a present need in the art for a non-invasive, low-cost, and low-risk means of early cancer detection.

### SUMMARY

The present disclosure provides methods and systems for diagnosis of diseases by performing absorbance spectroscopy in near- to mid-infrared (NIR-MIR) range of a patient sample and analyzing the resulting spectrometric signature to determine the presence of disease such as cancers.

According to one aspect of the present disclosure, a method for diagnosis of a disease includes analyzing a sample obtained from a patient by absorbance spectroscopy in the NIR-MIR range to produce a spectrometric signature. The spectrometric signature is received by a processor. The processor determines whether the spectrometric signature indicates the presence of the disease as a result and outputs the result. The method can optionally comprise administering a treatment to the patient to treat the disease when the result indicates that the disease is present.

According to another aspect of the present disclosure, a system for diagnosis of a disease's presence includes a memory and a processor configured to execute instructions stored in the memory in order to receive a spectrometric signature produced by absorbance spectroscopy in the near NIR and/or MIR range of a sample from a patient; determine whether the spectrometric signature indicates the presence of the disease as a result; and output the result.

In embodiments, the sample can be a lysate sample. The lysate sample can be obtained by adding a solubilizing or homogenizing solution to a blood serum sample. The solubilizing or homogenizing solution can be added in a 1:1 ratio with the blood serum sample. In embodiments, at least one of an optical binding solution or a proteolytic reagent is added to the lysate sample. In embodiments, combinations of any of solubilizing solutions, homogenizing solutions, optical binding solutions and proteolytic reagents can be added to the lysate sample.

Embodiments can further comprise drying the sample on an IR reflective sampling card. The card can be coated with aluminum or other non-IR-absorbing material.

In embodiments, the processor can determine whether the spectrometric signature indicates the presence of disease by providing the spectrometric signature to a computation engine, comprising a model architecture and one or more model parameters. The computation engine can execute a computation algorithm, such as a machine learning algorithm, configured to provide the result based on the spectrometric signature, the model architecture, and the one or more model parameters.

In embodiments, the computation engine can receive feedback indicating the correctness of the result. At least one model parameter can be updated based on the feedback.

According to another aspect of the present disclosure, a method of detection of disease agents in a sample is disclosed. The method includes receiving a patient whole blood sample in a coagulation cuvette, operating the coagulation cuvette to release a serum sample into an analysis chamber, inserting the cuvette into a spectrophotometer using near and/or mid infrared spectrum, determining whether a disease agent is present in the serum sample, and outputting a result indicating whether the disease agent was detected.

According to another aspect of the present disclosure, a system for detection of disease agents in a sample includes a coagulation cuvette, a spectrophotometer using near and/or mid infrared spectrum, and a processor associated with the spectrophotometer and configured to carry out analysis of a spectrophotometric signature output by the spectrophotometer.

According to another aspect of the present disclosure, a serum-separating cuvette includes a serum analysis chamber comprising a sample reservoir and one or more serum channels above the sample reservoir, a coagulation chamber comprising a coagulating agent to coagulate a sample introduced to the coagulation chamber, a clot strainer to remove clotted whole cells from the sample, and channel plugs. The channel plugs and the serum channels can form a releasable seal between the coagulating chamber and the analysis chamber such that sample serum may flow into the analysis chamber.

Embodiments of the present disclosure provide artificially intelligent and machine learning-driven optical molecular sensing systems and methods for detection of disease, including early cancer detection. Embodiments comprise a portable self-powered optical sensing device, a disposable sampling tube, or a microfluidic cassette (microcuvette), with an optical sensing cocktail solution. Data is collected and analyzed by an application with the predictive training database (local and cloud-based) on a smart device or a computer. The system can be applied for the analysis of cancer-specific biomolecules performed directly in serum. Only a relatively small volume of sample (˜50-200 μL) is needed for the biomolecular analysis, which takes 20 minutes or less to complete without damaging the sample.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures.

FIG. 1 is a flowchart depicting a method for detecting disease using a centrifuge and serum, according to an embodiment.

FIG. 2 is a flowchart depicting a method for detecting disease using a serum cocktail, according to an embodiment.

FIG. 3 is a flowchart depicting a method for detecting disease using a specialized cuvette, according to an embodiment.

FIG. 4 is a flowchart depicting a method for detecting disease using a lysate, according to an embodiment.

FIG. 5 is a schematic diagram depicting a computation engine, according to an embodiment

FIG. 6 is a schematic diagram depicting a method of training a computation engine, according to an embodiment.

FIGS. 7-10 are normalized spectra depicting differences in spectroscopic output.

FIGS. 11A and 11B are charts depicting example outputs, according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Non-invasive diagnosis and early detection of diseases such as cancer remain the major problems in health care today. New and emerging non-invasive tests that include biomarker/genetic tests, liquid biopsy, and breath biopsy are being developed. However, the high costs, lengthy testing procedures, and low positive test accuracy make them unfit for routine testing. The system and method of the present disclosure are designed to revolutionize liquid biopsy-based diagnostics that can be performed quickly and at low-cost directly in blood serum or plasma, eliminating the need for multiple lengthy steps of sample processing.

Liquid biopsy-based diagnostics can offer high sensitivity and accuracy for disease detection. Current liquid biopsy technologies for cancer diagnosis include the analysis of circulating tumor cells (CTC) and cell-free circulating tumor (ct) DNA, mRNA, and microRNA (miRNA) in blood samples. Others include the isolation and analysis of extracellular vesicular/exosomal biomarkers from plasma or serum. These technologies rely on the analysis of predefined sets of biomarkers based on the use of flow cytometry, next-generation sequencing polymerase chain reaction (PCR) and related chemistry-based detection, and protein/glycan detection. However, these technologies are still in their infancy, and their successes are limited by multiple factors.

First, due to cancer heterogeneity, no one liquid biopsy platform can consistently and reliably measure a predefined set of biomarkers for early cancer detection. Second, sample analyses that require multiple steps of preparation, handling, extended chemical reactions, detection, and data analysis are prone to technical and human errors, not to mention the high costs for technology development and utility in the clinical settings. Lastly, the current liquid biopsy technologies require a large sample volume for analyses, and the samples cannot be reused for multiple assays. The solution to these problems is an innovative non-destructive, broad molecular fingerprint-based liquid biopsy technology that goes straight from serum to detection and results within 10-30 minutes without lengthy sample processing. The samples can also be reused for multiple assays.

The optical sensing of the present disclosure is based on spectrophotometric analysis of cancer-specific molecular “fingerprints” generated by the complex mixture of biomolecules present in the serum. This technology works based on the principle that blood samples from patients with developing cancer contain biomolecules with different structural compositions than those from healthy individuals. These include differences in the biochemical components, proteins, circulating cell-free DNA/RNA/miRNA, and glycans (carbohydrates) that are both soluble in serum as well as packed inside the lipid encapsulated extracellular vesicles (EVs), such as exosomes, present in the serum.

Exosomes are small membrane-bound EVs secreted by tissue and immune cells for cell-to-cell communications, such as those in response to the presence of cancer. They are smaller than other EVs such as apoptotic bodies (released by dying cells) and microvesicles found in the serum, but they contain biological components that are crucial for cancer cells. More importantly, cancer cells generally secrete more exosomes and process more biomolecules than healthy cells. The overall cancer-associated exosomal contents are distinct from healthy exosomes because they are used to mediate communication between other cancer cells within the tumor microenvironment (or niche), for modulating and suppressing the immune response, and for establishing a niche supportive of cancer growth and spread. In addition, serum biomolecules, particularly the densely packed cargos inside the exosomes, form different molecular interactions and chemical bonds that result in molecular fingerprints unique to different physiological/pathological conditions.

Unlike other techniques that focus specifically on DNA, mRNA, miRNA, or the exosomes, the present disclosure detects the complex mixture of all components present in the serum and within the larger EVs and exosomes. This is beneficial, at least because cancer related components are also found in the larger microvesicles as well as in the apoptotic bodies of dying cancer cells. By measuring the biochemical and physical properties of the serum and EV components, the differences in the molecular fingerprints can be linked to the presence of different types of cancer. The fingerprints are based on spectrometric signatures using a broad-spectrum spectroscopy. This concept is well-supported by multiple studies reported in the literature and our own preliminary data. Using infrared (IR) spectroscopy, differences in serum molecular compositions can be documented to generate unique spectrometric signatures, or fingerprints, specific to different health conditions.

The present disclosure provides a fast, low-cost, and low-risk screening and monitoring for early signs of diseases such as cancer using a small amount of blood sample. Because the present disclosure can use optical sensing technology, the samples are not destroyed and can also be used for other tests without the need for additional blood draws. The cancer sensing strategy disclosed herein looks for the differences in a broad, biomarker-agnostic cancer fingerprints and combines with predefined cancer biomarker-specific signals, generating over 1,000 data points in one reading (˜2 sec per scan). This technique allows for early detection of different types of cancer from just one test, using one sample.

The technology of the present disclosure measures blood serum for molecular fingerprints using the visible and near-infrared (VIS/NIR) to mid-infrared (MIR) light absorbance spectra as the base component combined with the modulated NIR/MIR spectra and the predefined biomarker-specific fluorescence spectra. This allows sensing devices embodying the present disclosure to be compact, portable, and battery powered. In addition to the base spectrometric signatures and biomarker-specific signals that define the molecular fingerprints, we also measure the modulated absorbance spectra using specific chemical compounds, peptides, and antibodies designed to bind to specific sets of cancer-specific proteins, nucleic acids, and carbohydrates to produce broad molecular fingerprints for a multi-cancer detection platform and enhance early cancer detection accuracy.

Throughout this specification, wavelength or wavenumber ranges are referred to as “mid-IR” or “near-IR.” Depending upon the context, these terms can have different meanings within various technical disciplines. However, for purposes of this document, it should be understood that near-IR or NIR refers to light having a wavelength between about 600 and about 2500 nm, and in the majority of applications described herein between 600 and 1100 nm. Mid-IR or MIR refers to light having a wavelength range between about 1250 and about 25,000 nm, or in the majority of applications described herein between 2500 and 25,000 nm. “Infrared light source” refers to one or more optical sources that generate or emits radiation in the infrared wavelength range, For example it can comprise wavelengths within the mid-IR (2-25 microns). An infrared light source may generate radiation over a large portion of these wavelength sub-regions, or have a tuning range that is a subset of one of the wavelength ranges, or may provide emission across multiple discrete wavelength ranges, for example 2.5-4 microns, or 5-13 microns, for example. The radiation source may be one of a large number of sources, including thermal or Globar sources, supercontinuum laser sources, frequency combs, difference frequency generators, sum frequency generators, harmonic generators, optical parametric oscillators (OPOs), optical parametric generators (OPGs), quantum cascade lasers (QCLs), nanosecond, picosecond, femtosecond, and attosecond laser systems, CO2 lasers, heated cantilever probes or other microscopic heaters, and/or any other source that produces a beam of radiation. The source may be narrowband, for example with a spectral width of <10 cm-1 or <1 cm-1 less, or may be broadband, for example with a spectral width of >10 cm-1, >100 cm-1 or greater than 500 cm-1.

“Infrared absorption spectrum” refers to a spectrum that is proportional to the wavelength dependence of the infrared absorption coefficient, absorbance, or similar indication of IR absorption properties of a sample. An example of an infrared absorption spectrum is the absorption measurement produced by a Fourier Transform Infrared spectrometer (FTIR), i.e. an FTIR absorption spectrum. In general, infrared light will either be absorbed (i.e., a part of the infrared absorption spectrum), transmitted (i.e., a part of the infrared transmission spectrum), or reflected. Reflected or transmitted spectra of collected light can have a different intensity at each wavelength as compared to the intensity at that wavelength in the probe light source.

The terms “about” or “approximate” and the like are synonymous and are used to indicate that the value modified by the term has an understood range associated with it, where the range can be ±20%, ±15%, ±10%, ±5%, or ±1%.

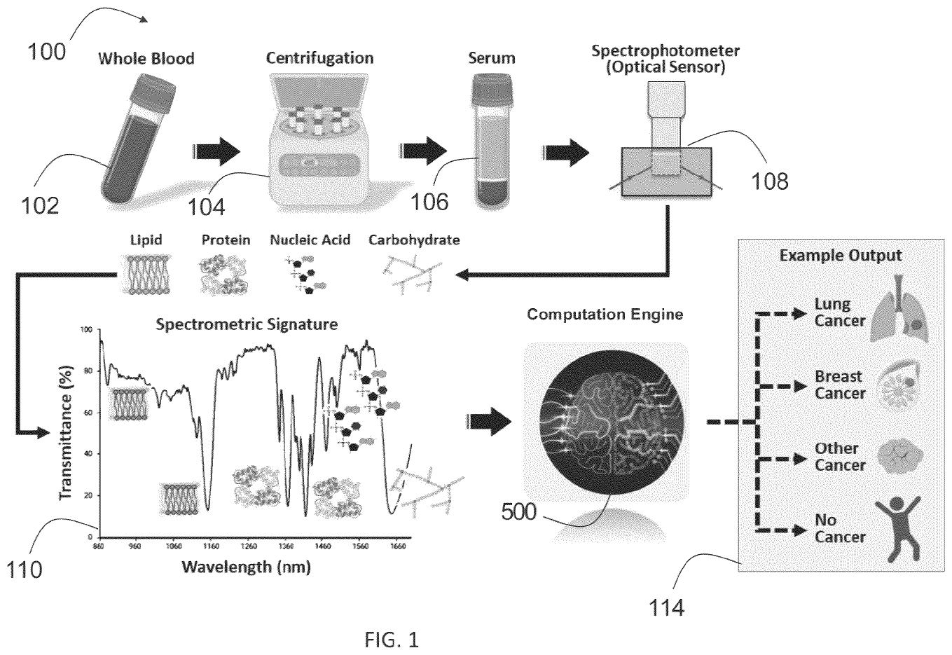

Embodiments of the present disclosure are directed to disease detection devices and procedures as depicted generally in the flowpath 100 of FIG. 1. The present disclosure focuses on cancer as the diagnosis of interest by way of example, the principles, devices, and methods of the present disclosure may be applicable to the diagnosis of other diseases, such as heart disease, diabetes, COVID-19, and others.

A whole blood sample 102 is taken from a patient and centrifuged 104 to separate out the blood serum 106. The serum is then optically analyzed 108 producing spectrometric signature 110, or “molecular fingerprint,” of the EVs and exosomes present in the patient's blood. This spectrometric signature contains unique peaks for each type of molecule present in the serum, including lipids, proteins, carbohydrates, and nucleic acids.

A whole blood sample 102 is described as the target sample in this and other examples herein, but in embodiments other samples may be used, including but not limited to cerebral-spinal fluid or other liquid biopsy, saliva, urine, etc. Solid or liquefied tissue or vapor, e.g. breath, analysis are also envisioned. Whole blood may generally be preferred due to the ease and non-invasive character of obtaining a whole blood sample, as well as blood's holistic representation of the cells of the body's various systems.

Centrifuging 104 the sample removes whole blood cells from the serum 106 to prevent the larger whole cells from obscuring EV's and exosomes. In embodiments, centrifuging the sample may be omitted so the whole blood sample may be analyzed by the spectrophotometer 108.

In embodiments, a sensor cocktail solution may be added to the serum 106 prior to the analysis by optical sensor 108. The sensor cocktail solution is a mixture of antibodies, peptides, and/or molecular binding reagents against cancer type-specific proteins and biomolecules, including EGF, INHBA, CD44s, and other extracellularly secreted/released molecules present in the serum linked to different types of cancer. The antibodies and peptides are unconjugated for the measurement of modulated absorbance spectra, or labeled with fluorescence resonance energy transfer (FRET) fluorophores for the analysis of fluorescence spectra. Addition of the sensor cocktail modifies the output signature according to whether the target molecules are present, providing a presence/absence detection of whether cancer-characteristic molecules are present in the sample. Though targeting antibodies may be tagged by any means available in the art, such modification is unnecessary for target identification under the principles and methods of the present disclosure.

A variety of possible combinations for the cocktail solution are envisioned, assembled from various antibodies, peptides, and molecular binding reagents known in the art. For example, a general cancer detection solution may identify a broad array of EVs and exosomes associated with various cancer lineages to provide a screen measure for whether more targeted testing is appropriate. Other cocktail solutions may be specifically tailored to cellular products associated with a particular cancer lineage, e.g., lung cancer, breast cancer, etc., or may be tailored such that a particular cancer lineage will produce a characteristic output.

The sample may be analyzed by a spectrophotometer 108 to yield a spectrometric signature 110. Any broad-spectrum spectroscopy frequency may be used, but in certain preferred embodiments, the infrared (IR), near-infrared (NIR), or visible (VIS) light spectrums may be used. The shorter wavelengths of these ranges may be preferred to enable a smaller, more compact, spectrophotometer to be used, which may additionally increase portability of the necessary equipment and improve mobility and availability of the testing. Shorter wavelength spectrophotometers may also be preferred due to their lower power requirements and simpler sample preparation, as aqueous samples may be analyzed.

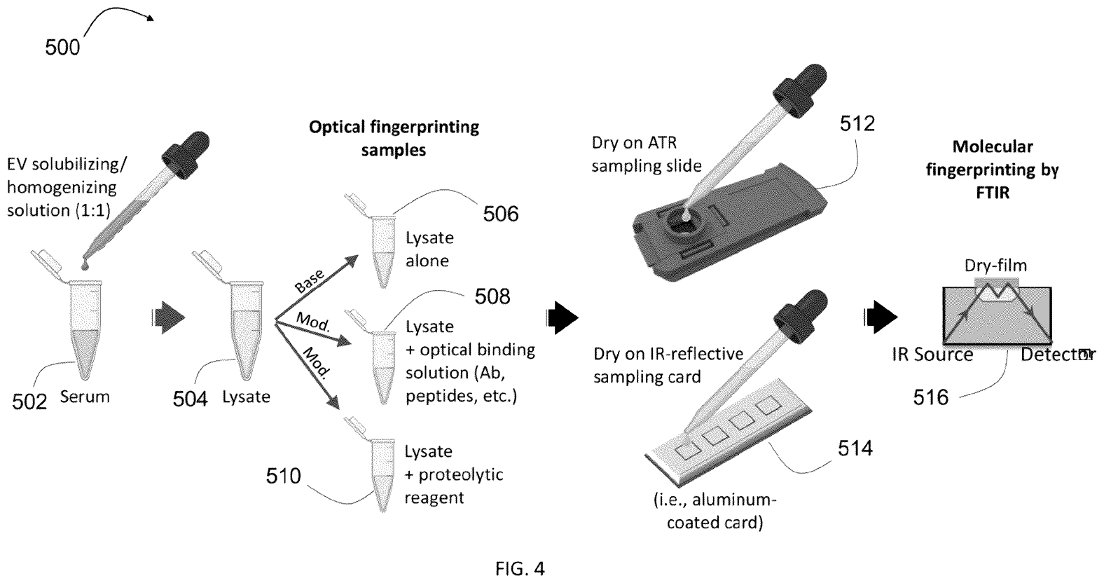

Despite these advantages offered by NIR or MIR ranges, the spectrometric signatures 110 produced are often complex, due to the broad overtone and combination bands produced by NIR or MIR absorbance. As discussed in more detail with respect to FIG. 4 below, computation engine 500 can be configured to receive a spectrographic signature 110 and automatically one or more interpretations of the spectrometric signature 110.

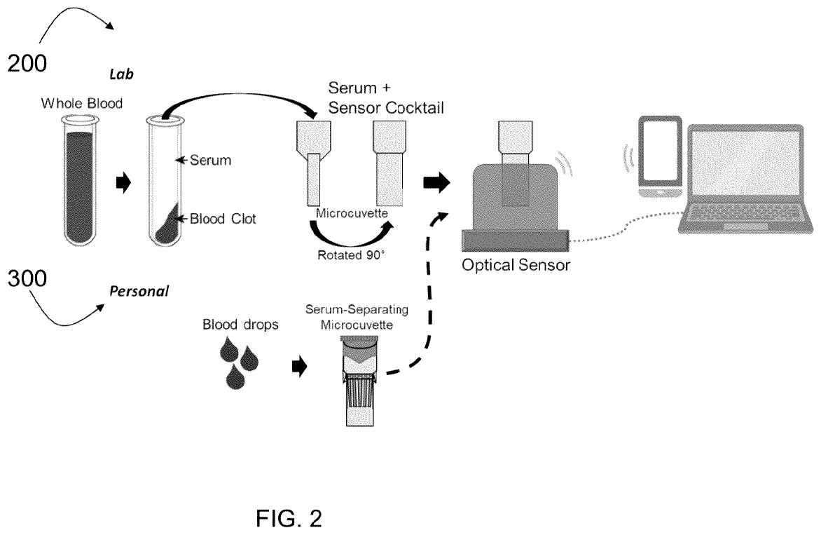

FIG. 2 is a schematic diagram depicting possible modes of sample collection and preparation, according to embodiments.

A first system 200 can comprise a rapid blood-coagulating microcentrifuge tube for blood collection and a high-speed microcentrifuge for serum separation, an optically clear microcuvette for serum and sensor cocktail mix, a Bluetooth/Wi-Fi connected optical sensor, and a computer or a smart device with a software application which can include, or be communicatively connected to, computation engine 500. System 200 can therefore provide a lab-based analysis system.

A second system 300 can comprise a serum-separating microcuvette designed for sampling blood drops from a finger stick that can be done at home for personal use, or in other non-laboratory settings. Serum collected can be mixed with preassembled sensor cocktail and analyzed in a portable optical sensor or be sent to a testing facility for a more comprehensive analysis. System 300 can therefore provide a more portable analysis system, such as a home-based system, or a mobile system.

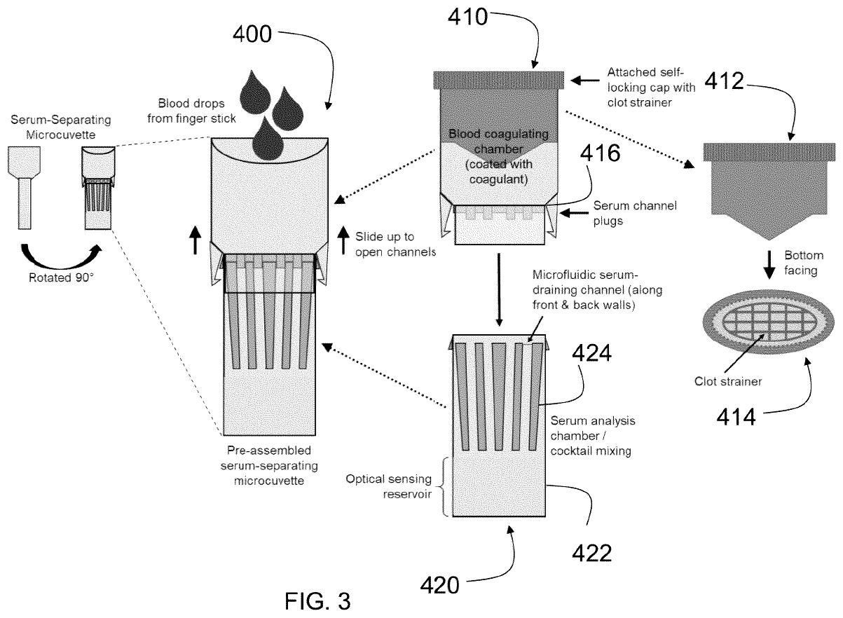

FIG. 3 is a schematic view depicting a serum separating microcuvette as provided by embodiments. A patient or other user may have a small, low power spectrophotometer, such as a NIR or VIS spectrophotometer, either individually owned or available in a public place such as a pharmacy or a clinic. Such a machine for individual or public use may be designed to directly receive and analyze the serum-separating microcuvette. A user may screen themselves for cancer or other diseases of concern by performing a finger prick 302 and placing a few drops of whole blood into the serum-separating microcuvette 304. The serum-separating microcuvette may be placed in the optical sensor 306 and analyzed by the associated processor 308. An output result is generated 310 and may be presented to the user themselves, or the system may be configured to permit the user to send the results directly to a care provider, such as by providing appropriate contact for the provider or by integration with an electronic medical record system.

Serum separation microcuvette 400 comprises a blood coagulating chamber 410 and a serum analysis chamber 420. In embodiments, microcuvette 400 may be pre-assembled to support its function of ease of use. Blood coagulant chamber 410 rests atop of serum analysis chamber 420. A user may place a few drops of blood, e.g., from a finger stick, into the upper coagulating chamber 410. Coagulating blood cells are captured in the coagulating chamber 410 and serum is able to run down, or gravity drain, into the lower serum analysis chamber 420.

Blood coagulating chamber 410 comprises a self-locking cap 412, a clot strainer 414, and serum channel plugs 416. Coagulating chamber 410 is coated with a coagulating agent, so that blood, or other biological sample, begins to coagulate when added to the chamber. The self-locking cap 412 may be configured, for example by an interference fit, to permanently seal the open top of the coagulation chamber 410. The bottom of the coagulating chamber may be releasably sealed, such as by channel plugs 416. Clot strainer 414 may be integrated with cap 412, or other oriented above channel plugs 416, and serve to capture clotted cells so that serum may be released into the lower analysis chamber 420. Channel plugs 416 may retain the sample serum and cells within the coagulating chamber 410 until sufficient cells have been removed by the strainer 414 to yield a satisfactory serum sample. Plugs 416 may be released, such as by lifting or twisting the coagulating chamber 410, to allow the serum to flow into the lower analysis chamber 420.

Analysis chamber 420 comprises reservoir 422 and draining channel 424. Draining channels 424 in the upper portion of analysis chamber 420 can direct serum to the lower reservoir 422 and provide some additional straining of the sample. Channels 424 may also participate in the releasable seal between coagulation chamber 410 and analysis chamber 420. For instance, channels 424 may interlock with plugs 416 of the coagulating chamber to prevent the sample from entering the analysis chamber 420 until the plugs 416 are released, e.g., by lifting the coagulating chamber 410 to remove plugs 416 from between channels 424. Serum may be analyzed, such as by spectroscopy, once collected in reservoir 422. In embodiments, analysis chamber may also serve to enable cocktail mixing, by the addition of targeting antibodies or peptides on channels 424 or directly within reservoir 422 such that the serum mixes with the targeting agents as it collects in the reservoir 422. At least reservoir 422, and in embodiments all or any components of cuvette 400, are formed of an optically clear material.

In embodiments the methods of the present disclosure may be used with a lysate as depicted generally in the flowpath 500 of FIG. 4. A blood serum sample 502 is prepared by taking a whole blood sample from a patient and centrifuging it to separate out the red blood cells. An EV solubilizing and homogenizing solution is then added to the blood serum 502 in a 1:1 or the most optimal ratio to lyse EV's in blood serum 502 and produce a lysate 504. A base sample 506 consisting of the lysate alone may be used for optical fingerprinting, or the lysate 504 may be modified before optical fingerprinting. A first modified sample 508 can include an optical binding solution that is added to the lysate for optical fingerprinting. The optical binding solution may include antibodies, peptides, and other compounds against cancer type-specific proteins and biomolecules. A second modified sample 510 can include a proteolytic reagent added to the lysate 504. The proteolytic reagent may break down peptides in the lysate into smaller peptides for optical fingerprinting. In some embodiments, combinations of two or more of the lysate alone, the lysate with optical binding solution, and lysate with proteolytic reagent may be used as optical fingerprinting samples.

The optical fingerprinting samples may then be applied to and dried on an attenuated total reflection (“ATR”) crystal 512 or an infrared (“IR”)-reflective sampling card 514. Once the optical fingerprinting samples have dried, Fourier-transform infrared spectroscopy (FTIR) is performed 516 using systems and methods known in the art. When an ATR crystal is subjected to FTIR, an IR beam is passed into the ATR crystal with a defined refraction index and through the sample on a dry film. The IR beam will be refracted back into the crystal and a detector will detect the resulting wavelength and produce a spectrometric signature.

In some embodiments, the optical fingerprinting sample is dried on an IR-reflective sampling card 514, such as an aluminum-coated card. Aluminum is highly reflective and will reflect an IR beam, and other coatings are contemplated such as those containing on or more materials such as gold, indium tin oxide, zinc oxide, or the like. The coated IR-reflective sampling card is then subjected to FTIR 516, and the IR beam will pass through the card and dried sample and reflect the IR beam to be detected by the detector, which will produce a spectrometric signature.

Blood serum analysis with the systems and methods of the present disclosure can produce results within 30 minutes or less, 20 minutes or less, 10 minutes or less, depending on the system configuration. The present disclosure may advantageously be used in a low-resource setting using an integrated system consisting of an optical sensor device, either a microcentrifuge or a serum-separating microcuvette, and a smartphone or a laptop computer. After separation from blood clots, a serum sample placed inside a microcuvette is analyzed using the optical sensor device to perform the base and modulated absorbance spectroscopy. Small samples may provide definitive results using the methods and systems disclosed herein, for example, samples of 50 μl, 60 μl, 70 μl, 80 μl, or less may be effectively used.

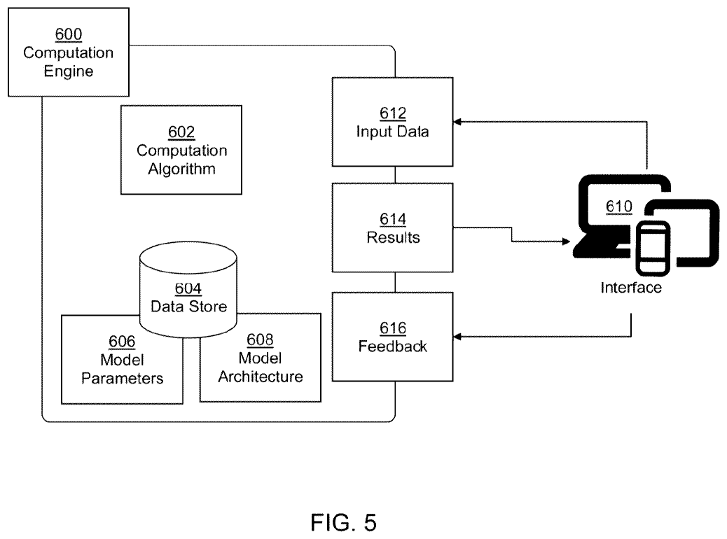

FIG. 5 is a schematic diagram depicting components of computation engine 600, according to an embodiment. In embodiments, computation engine 600 can use one or more computational techniques including artificial intelligence techniques such as machine learning to enable efficient interpretation of the spectrographic signature.

Many different machine learning algorithms exist and, in general, a machine learning algorithm seeks to approximate an ideal target function, ƒ, that best maps input variables x (the domain) to output variables y (the range), thus:

y=ƒ(x)

The machine learning algorithm as an approximation of ƒ is therefore suitable for providing predictions of y. Supervised machine learning algorithms generate a model for approximating ƒ based on training data sets, each of which is associated with an output y. Supervised algorithms generate a model approximating ƒ by a training process in which predictions can be formulated based on the output y associated with a training data set. The training process can iterate until the model achieves a desired level of accuracy on the training data.

Other machine learning algorithms do not require training. Unsupervised machine learning algorithms generate a model approximating ƒ by deducing structures, relationships, themes and/or similarities present in input data. For example, rules can be extracted from the data, a mathematical process can be applied to systematically reduce redundancy, or data can be organized based on similarity. Semi-supervised algorithms can also be employed, such as a hybrid of supervised and unsupervised approaches.

Notably, the range, y, off can be, inter alia: a set of classes of a classification scheme, whether formally enumerated, extensible or undefined, such that the domain x is classified e.g. for labeling, categorizing, etc.; a set of clusters of data, where clusters can be determined based on the domain x and/or features of an intermediate range y′; or a continuous variable such as a value, series of values or the like.

Regression algorithms for machine learning can model ƒ with a continuous range y. Examples of such algorithms include Ordinary Least Squares Regression (OLSR); Linear Regression; Logistic Regression; Stepwise Regression; Multivariate Adaptive Regression Splines (MARS); and Locally Estimated Scatterplot Smoothing (LOESS).

Clustering algorithms can be used, for example, to infer ƒ to describe hidden structure from data including unlabeled data. Such algorithms include, inter alia: k-means; mixture models; neural networks; and hierarchical clustering. Anomaly detection algorithms can also be employed.

Classification algorithms address the challenge of identifying which of a set of classes or categories (range y) one or more observations (domain x) belong. Such algorithms are typically supervised or semi-supervised based on a training set of data. Algorithms can include, inter alia: linear classifiers such as Fisher's linear discriminant, logistic regression, Naïve Bayes classifier; support vector machines (SVMs) such as a least squares support vector machine; quadratic classifiers; kernel estimation; decision trees; neural networks; and learning vector quantization.

Computation engine 600 can be a discrete software module in that it is individual, separate and/or distinct and can be portable in the sense that computation engine 600 can be stored or transmitted for execution in potentially multiple execution environments such as physical or virtual computer systems or software platforms executing in a computer system such as runtime environments, operating systems, platform software and the like.

Computation engine 600 can include an executable computation algorithm 602 such as any of the machine learning algorithms hereinbefore described or other suitable machine learning algorithms as will be apparent to those skilled in the art. Suitable machine learning algorithms are configurable to execute within the scope of the computation engine 600 on the basis of input parameters including, for example, domain data and/or configuration parameters as an input for the algorithm to generate a machine learning result such as range data and/or other output data. For example, computation algorithm 602 can be provided as a method of a software object or a subroutine, procedure or function in a software library. Thus, the machine learning algorithm is executable to perform machine learning functions including any or all of a training phase of operation for training the computation engine 600 where the algorithm is supervised or semi-supervised; and/or a processing phase of operation of the algorithm tor providing one or more machine learning results.

Computation engine 500 further can further comprise a data store 604 for the storage of data by the algorithm. The data store can be a volatile or non-volatile storage such as a memory. The data store 604 can be used for the storage of data required by the algorithm such as machine learning parameters 606, machine learning data structures including representations of machine learning models 608 such as, inter alia: tree data structures; representations of regression analysis data structures; representations of neural network data structures; variables; and any other data that may be stored by the machine learning algorithm as will be apparent to those skilled in the art. Thus, in this way, computation engine 600 provides a discrete encapsulation of one or more machine learning algorithms and the data required for or by such algorithms.

Computation engine 600 can further include an interface 610 for communication external to the computation engine 500 for receiving inputs and delivering outputs. For example, machine learning parameters including configuration information, training data and machine learning input (domain) information can be communicated via the input as at least part of input data 612. Results 614 such as predictions generated by the model can be communicated as output via the interface. Feedback 616 can comprise additional inputs that can be used during a discrete operation of training of computation engine 600 or provided as input during regular operation of computation engine 600 to provide iterative learning.

The interface 610 can receive user inputs and provide user outputs regarding configuration of computation engine 600. The interface 610 can comprise a mobile application, web-based application, or any other executable application framework. The interface can reside on, be presented on, or be accessed by any computing devices capable of communicating with the various components of computation engine 600, receiving user input, and presenting output to the user. In embodiments, the interface can reside or be presented on a smartphone, a tablet computer, laptop computer, or desktop computer.

Computation engine 600 can implement any appropriate machine learning algorithm or architecture having any number of layers such as convolutional layers, activation layers, pooling layers, and the like. Furthermore, computation engine 600 can be trained by any appropriate training method. In one embodiment, computation engine 600 can comprise a single-layer neural network and can be trained using supervised training techniques.

Training may be by targeting to known molecules and providing spectrums of known diagnoses and controls. In embodiments, results may be normalized to total absorbance prior to analysis to increase overall clarity and consistency of results.

In one embodiment the training data can include spectrometric signatures 110, which can be labeled with known diagnoses, such as: healthy (no cancer), breast cancer, lung cancer, other cancer, or combinations thereof. Computation engine 500 can therefore classify a given spectrographic signature input as indicating a diagnosis (with the likelihood thereof).

The training data can also include spectrometric signatures 110 labeled based on the known presence (or lack thereof) of molecules of interest (such as lipids, proteins, etc.). Computation engine can therefore classify a given spectrometric signature input as indicating the presence of one or more molecules of interest (with the likelihood thereof).

In addition to spectrometric signatures 110, in embodiments inputs to computation 112 (whether in training data, or in data to be evaluated), can include additional information about the patient including demographic data (age, ethnicity, etc.), health characteristics (height, weight, medical history, medications, etc.) and the like. As such computation engine 600 can use additional patient data to determine outputs.

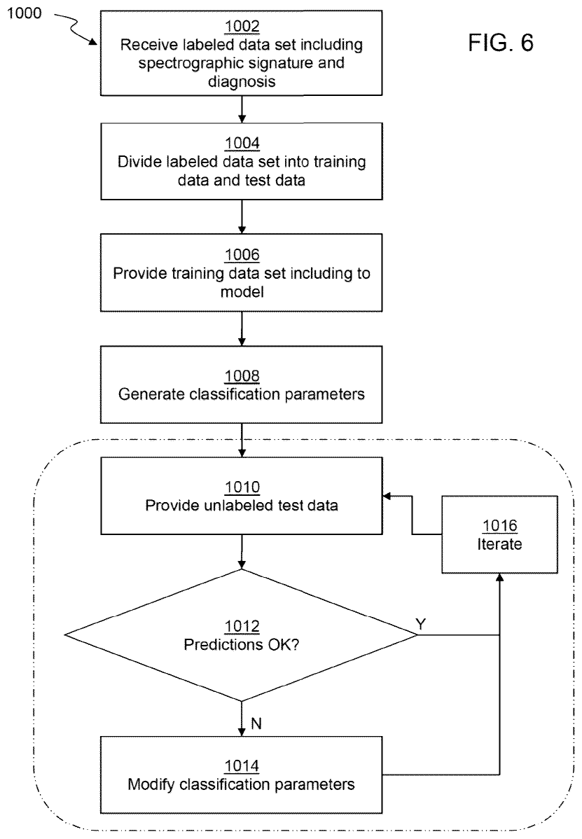

FIG. 6 is a flowchart depicting a method 1000 for training computation engine 600. At 1002, a labeled data set spectrometric signatures 110 can be received or provided, as discussed above the data set can further include additional patient data. At 1004, the labeled data set can be divided into training data and test data, for example, the training data set can comprise a certain percentage, such as 60% of the entries in the labeled data set. At 1006, the training data set, including the associated known diagnoses, molecule presence, or other factors can be provided to the model. At 1008, the model can generate a first set of parameters by any appropriate method.

At 1010, some or all of the testing data set can be provided to the model, without the known entries. At 1012, predictions generated by the model can be compared to the known entries for each input in the test data set. If, at 1012, the recommendations show and acceptable amount of error, additional testing can occur by iterating at 1016. If, at 1012, the recommendations show an unacceptable amount of error, the classification parameters can be modified before proceeding to iterate at 1016. This testing phase can be iterated until the test data set is exhausted, until the amount of error consistently hits a minimum threshold, for a set period of time, or until some other criteria is met.

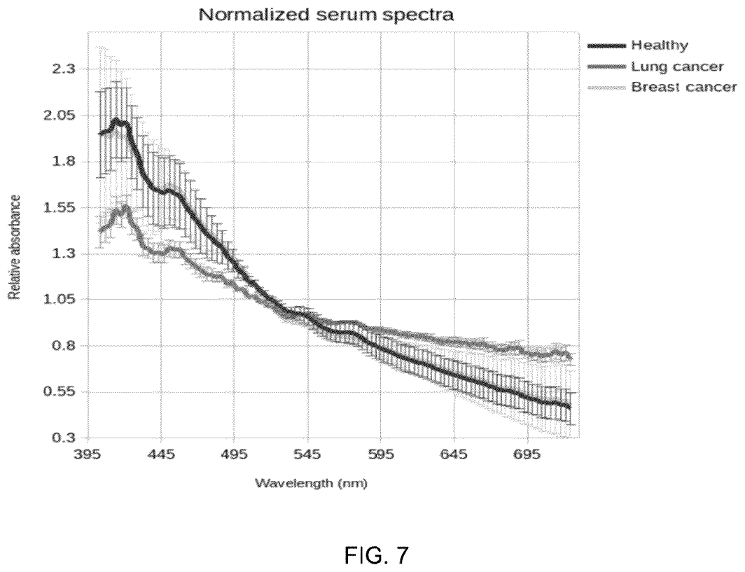

FIGS. 7-10 are charts depicting example as recorded by embodiments of the present disclosure as detailed below.

### Example 1: Proof of Concept

A 200 μl sample of serum per patient collected by centrifugation of whole coagulated blood. Spectroscopy was performed to measure the base and modulated absorbance spectra at a range of 403 nm to 724 nm with a resolution (interval) of ˜3 to 4 nm. Full absorbance spectrum measurement was performed continuously for ˜5-10 seconds, or until the absorbance signals stabilized (usually <20 seconds). The final absorbance spectrum for each sample was averaged across the total measurement time. Averaged base absorbance spectra of serum samples from four healthy (non-cancer), three lung cancer, and two breast cancer patients were normalized to the total absorbance value individually for each sample and plotted for visualization (FIG. 7). Distinct spectra were observed between healthy and lung cancer patients, while the difference between healthy and breast cancer patients was less evident but discernable.

### Example 2: Specificity

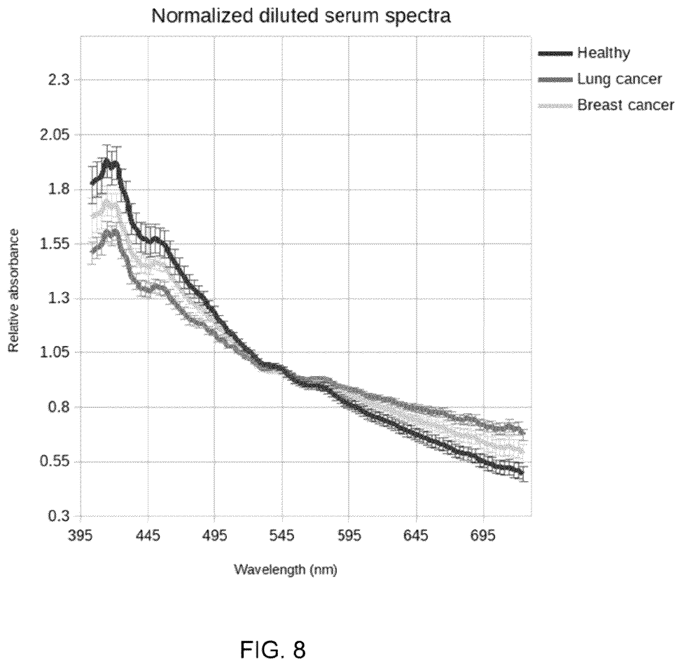

To determine whether cancer patient serum spectroscopy was a measurement of cancer-associated molecular fingerprints, lung and breast cancer serum sample were mixed with six different healthy serum conditions. If the absorbance spectra observed were dependent on the overall serum contents irrespective of cancer, then cancer-specific spectroscopy would be masked by non-cancer healthy serum components and render indistinguishable spectra between the healthy and cancer samples. To increase serum complexity, two healthy serum samples at a ratio of 1:1 were mixed to generate six different serum combinations. Each cancer serum sample was mixed with each healthy serum mixture at a ratio of 1:1, producing 18 lung and 12 breast cancer samples at a 50% dilution for each sample. Cancer samples diluted by 50% in different combinations of healthy sera produced more distinct absorbance spectra compared to the six different healthy serum combinations (FIG. 8). These results indicate that the molecular fingerprint spectroscopy was specific to lung and breast cancer samples tested.

### Example 3: Modulation

Whether the absorbance spectra could be modulated using an anti-human epidermal growth factor (EGF) antibody was tested. EGF is an important growth factor produced by the cancer cells for tumor formation and is present at higher levels in the serum from cancer patients. The hypothesis was that anti-EGF antibody would bind to EGF protein present in the cancer patient serum and change its overall molecular bonding, resulting in changes in the protein's light absorbance spectrum. To prove this hypothesis, healthy and lung cancer serum were mixed with 1 μg of antibody (at a total of 1% by volume) and incubated for 0, 10, and 20 minutes at ambient temperature.

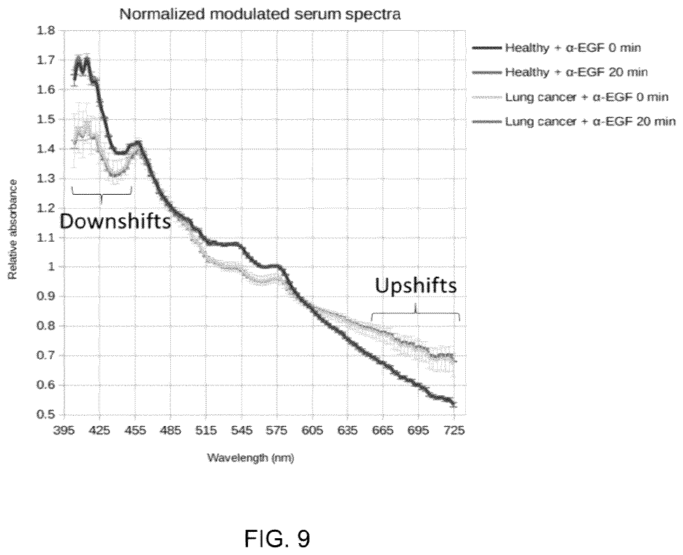

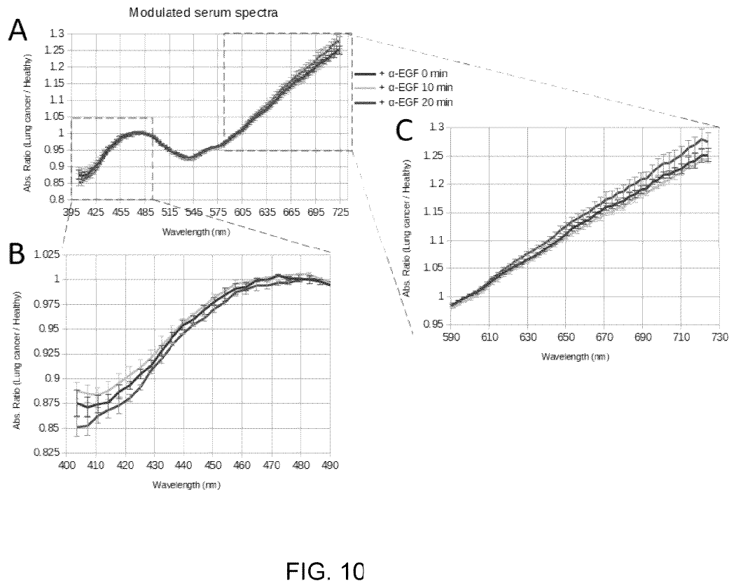

At 10 (data not shown) and 20 min incubation times, a slight increase in healthy serum absorbance spectrum was found at between 403 nm and 425 nm, while lung cancer serum absorbance was differentially modulated at different wavelengths (FIGS. 9 and 10 chart A). Lung cancer serum absorbance was upshifted at 10 min incubation and was downshifted after 20 min incubation at the shorter wavelength region (FIG. 10 chart B). In contrast to the 0 and 10 min incubation times, the spectrum was upshifted in the longer wavelength region after 20 min in the presence of EGF antibody (FIG. 10 chart C). These results indicate that the increased light absorbance at the shorter wavelengths was due to the presence of unbound antibodies. When bound to EGF proteins in cancer patient serum, the antibody increased the light absorbance at the longer wavelengths, producing a modulated absorbance spectrum. The increase in the absorbance appeared to correlate with the increase in wavelength. As part of our technology development, spectroscopy will be performed beyond 724 nm up to 950 nm or longer to determine whether further increases in the absorbance will be observed.

### Example 4: Differentiation

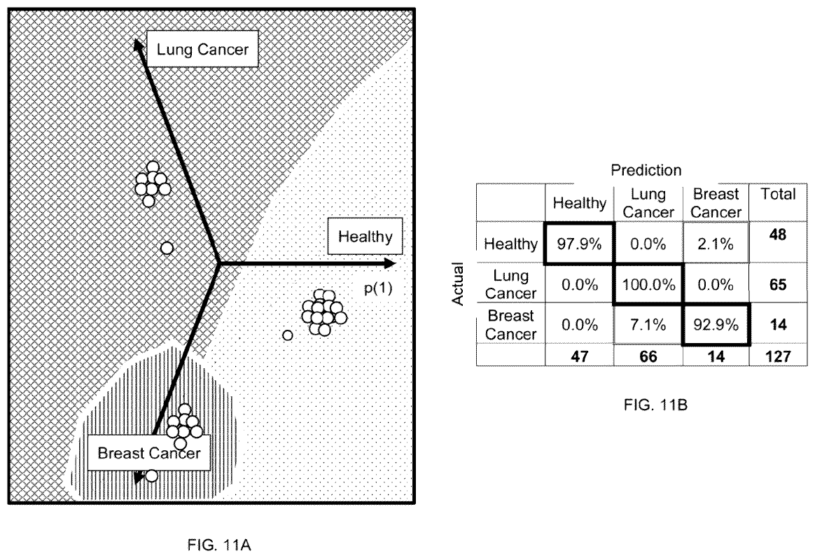

To show whether the system of the present disclosure can differentiate lung cancer from breast cancer and healthy serum samples, a neural network machine learning algorithm was used to train and build a predictive model using normalized data from serum spectroscopy. All samples tested, including undiluted and diluted (up to 64-fold) samples, were included in the leave-one-out cross-validation, where one sample was held back for testing, and the rest of the samples were used for training the model. The process of cross-validation was repeated until all the samples were tested. The results of machine learning cross-validation are visualized in FIG. 11A. The predictive model was able to accurately classify healthy samples (representing specificity) at 97.9%, lung cancer at 100%, and breast cancer at 92.9% (FIG. 11B) irrespective of sample dilutions. More breast cancer samples can be added to ensure sufficient training and to improve prediction accuracy.

In one embodiment, the system 100 and/or its components or subsystems can include computing devices, microprocessors, modules and other computer or computing devices, which can be any programmable device that accepts digital data as input, is configured to process the input according to instructions or algorithms, and provides results as outputs. In one embodiment, computing and other such devices discussed herein can be, comprise, contain or be coupled to a central processing unit (CPU) configured to carry out the instructions of a computer program. Computing and other such devices discussed herein are therefore configured to perform basic arithmetical, logical, and input/output operations.

Computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of diagnosis for a disease comprising:
obtaining a cell free sample comprising extracellular vesicles;
lysing the cell free sample to create a lysate sample;
adding at least one of an optical molecular binding solution or a proteolytic reagent to the lysate sample to create a processed lysate sample;
analyzing the processed lysate sample by absorbance spectroscopy in the near- and/or mid-infrared range to produce a spectrometric signature, wherein analyzing the processed lysate sample comprises:
receiving, by a processor, the spectrometric data corresponding to the processed lysate sample;
determining, by the processor, whether the spectrometric data corresponding to the processed lysate sample indicates the presence of the disease as a result; and
outputting, by the processor, the result.

2. The method of claim 1, further comprising administering a treatment to a patient to treat the disease when the result indicates that the disease is present.

3. (canceled)

4. A method of diagnosis for a disease comprising:
obtaining a cell free comprising extracellular vesicles from a blood serum sample; lysing the cell free sample to create a lysate sample, obtained by adding a solubilizing or homogenizing solution to the cell free sample;
adding at least one of an optical molecular binding solution or a proteolytic reagent to the lysate sample to create a processed lysate sample;
analyzing the processed lysate sample by absorbance spectroscopy in the near- and/or mid-infrared range to produce a spectrometric signature, wherein analyzing the processed lysate sample comprises:
receiving, by a processor, the spectrometric data corresponding to the processed lysate sample;
determining, by the processor, whether the spectrometric data corresponding to the processed lysate sample indicates the presence of the disease as a result; and
outputting, by the processor, the result.

5. The method of claim 3, wherein the solubilizing or homogenizing solution is added in a 1:1 ratio with the blood serum sample.

6. (canceled)

7. The method of claim 1, further comprising drying the sample on an IR-reflective or non-IR-absorbing sampling card.

8. The method of claim 5, wherein the IR-reflective sampling card is coated with aluminum.

9. The method of claim 1, wherein determining, by the processor, whether the processed lysate spectrometric signature indicates the presence of the disease as a result comprises:
providing the processed lysate spectrometric signature to a computation engine comprising a model architecture and one or more model parameters; and
executing, by the computation engine, a computation algorithm configured to provide the result based on the processed lysate spectrometric signature, the model architecture, and the one or more model parameters.

10. The method of claim 7, further comprising:
providing feedback indicating the correctness of the result to the computation engine; and
updating the one or more model parameters based on the result, the processed lysate spectrometric signature, and the feedback.

11. A system for diagnosis of a disease's presence, the system comprising:
a sample tube configured to hold a sample comprising extracellular vesicles (EVs);
an EV solubilizing and homogenizing solution configured to lyse the EVs to form a lysate sample;
at least one of an optical molecular binding solution or a proteolytic reagent configured to modify the lysate sample;
a memory; and
a processor configured to execute instructions stored in the memory in order to:
analyze the processed lysate sample by absorbance spectroscopy in the near- and/or mid-infrared range to produce a spectrometric signature, wherein analyzing the processed lysate sample comprises:
receiving, by the processor, spectrometric data corresponding to the processed lysate sample;
determining, by the processor, whether the spectrometric data corresponding to the processed lysate sample indicates the presence of the disease as a result; and
outputting, by the processor, the result.

12. The system of claim 9, wherein the processed lysate sample is derived from a blood serum sample.

13. The system of claim 9, wherein the processed lysate sample is derived from a whole blood sample.

14. (canceled)

15. The system of claim 11, wherein the lysate sample is obtained by adding a solubilizing or homogenizing solution to a blood serum sample.

16. The system of claim 12, wherein the solubilizing or homogenizing solution is added in a 1:1 ratio with the blood serum sample.

17. The system of claim 9, wherein the processor is configured to determine whether the
processed lysate spectrometric signature indicates the presence of the disease by:
providing the processed lysate spectrometric signature to a computation engine, comprising a model architecture and one or more model parameters; and
executing, by the computation engine, a computation algorithm configured to provide the result based on the processed lysate spectrometric signature, the model architecture, and the one or more model parameters.

18. The system of claim 9, wherein the processor is further configured to execute instructions in the memory in order to:
provide feedback indicating the correctness of the result to the computation engine; and update the one or more model parameters based on the result, the processed lysate spectrometric signature, and the feedback.

19. A method of detection of disease agents in a sample comprising:
receiving a patient whole blood sample in a coagulation cuvette;
operating the coagulation cuvette to release a serum sample into an analysis chamber fluidically coupled to the coagulation cuvette;
mixing the serum sample with at least one targeting agent within in the analysis chamber to create a processed serum sample;
inserting the cuvette into a spectrophotometer configured for near- and/or mid-infrared spectrum analysis;
determining whether one or more disease agents are present in the processed serum sample; and
outputting a result indicating whether the disease agents were detected.

20. A system for detection of disease agents in a sample comprising:
a coagulation cuvette;
a spectrophotometer configured for near- and/or mid-infrared spectrum analysis; and
a processor associated with the spectrophotometer and configured to carry out an analysis of a spectrophotometric signature output by the spectrophotometer by:
receiving a patient whole blood sample in a coagulation cuvette;
operating the coagulation cuvette to release a serum sample into analysis chamber fluidically coupled to the coagulation cuvette;
mixing the serum sample with at least one targeting agent within the analysis chamber to create a processed serum sample;
inserting the cuvette into a spectrophotometer configured for near- and/or mid-infrared spectrum analysis;
determining whether one or more disease agents are present in the processed serum sample; and outputting a result indicating whether the disease agents were detected.

21. A serum-separating cuvette comprising:
a serum analysis chamber comprising a sample reservoir and one or more serum channels above the sample reservoir;
a coagulation chamber comprising a coagulating agent to coagulate a sample introduced to the coagulation chamber, a clot strainer to remove clotted whole cells from the sample, and channel plugs;
wherein the channel plugs and the serum channels form a releasable seal between the coagulating chamber and the analysis chamber such that sample serum may flow into the analysis chamber.
